(19) United States
(12) Patent Application Publication
(10) Pub. No.: US 2026/0240646 A1
(43) Pub. Date: Aug. 20, 2026

(54) PROSTHETIC HEART VALVE

(71) Applicant: Medtronic, Inc., Minneapolis, MN (US)

(72) Inventor: Niall F. DUFFY, Galway (IE)

(21) Appl. No.: 19/165,342

(22) PCT Filed: Mar. 11, 2024
(86) PCT No.: PCT/IB2024/052345

§ 371 Date: Sep. 15, 2025

Related U.S. Application Data
(60) Provisional application No. 63/454,502, filed on Mar. 24, 2023.

(51) Int. Cl. A61F 2/24

(52) U.S. Cl. CPC A61F 2/2418; A61F 2/2436; A61F 2230/0006; A61F 2250/0007; A61F 2250/0037

(57) ABSTRACT

A transcatheter heart valve prosthesis includes an annular frame including a plurality of struts. The plurality of struts define a first plurality of cells arranged along a first cell axis and a second plurality of cells arranged along a second cell axis. First cell is aligned with a first ostia and a second cell is aligned with a second ostia. A prosthetic valve is attached to the frame and includes a plurality of leaflets and a commissure. Methods of rotationally aligning a transcatheter heart valve prosthesis within a native heart valve are provided.

### CROSS-REFERENCE TO RELATED APPLICATION

This application claims the benefit of U.S. Provisional Patent Application Ser. No. 63/454,502, filed Mar. 24, 2023, the entire content of which is incorporated herein by reference.

### FIELD

The present disclosure relates generally to a prosthetic heart valve assembly and, more particularly, to a heart valve prosthesis configured to avoid obstruction of the coronary arteries.

### BACKGROUND

It is known to provide a prosthetic heart valve assembly for implanting a heart valve prosthesis within a target site of the vasculature of a patient. The heart valve prosthesis can be moved from a radially-contracted position to a radially-expanded position. However, aligning the heart valve prosthesis to avoid obstructing the coronary arteries can be difficult.

### SUMMARY

The following presents a simplified summary of the disclosure to provide a basic understanding of some aspects described in the detailed description.

In aspects, a transcatheter heart valve prosthesis comprises an annular frame extending along a longitudinal axis between an inflow end of the transcatheter heart valve prosthesis and an outflow end of the transcatheter heart valve prosthesis. The annular frame comprises a plurality of struts and is configured to be adjustable between a radially-collapsed position and a radially-expanded position. The plurality of struts define a first plurality of cells arranged along a first cell axis that is substantially parallel to the longitudinal axis. The first plurality of cells comprise a first cell at a first axial location. The first cell is configured to be aligned with a first ostia such that a first radial axis intersects the longitudinal axis and passes through the first cell and the first ostia. A second plurality of cells is arranged along a second cell axis that is substantially parallel to the longitudinal axis. The second plurality of cells is spaced circumferentially apart from the first plurality of cells. The second plurality of cells comprise a second cell at the first axial location. The second cell is configured to be aligned with a second ostia such that a second radial axis intersects the longitudinal axis and passes through the second cell and the second ostia. A prosthetic valve is attached to the frame and comprises a plurality of leaflets. The prosthetic valve comprises a commissure where a first pair of leaflets of the plurality of leaflets are attached. The commissure is attached to the frame and lies within a third cell axis that is substantially parallel to the longitudinal axis. The third cell axis is positioned circumferentially between the first cell axis and the second cell axis such that a first separating distance circumferentially separates the first cell axis from the third cell axis and a second separating distance circumferentially separates the second cell axis from the third cell axis. The first separating distance is different than the second separating distance.

In aspects, the prosthetic valve further comprises a second commissure where a second pair of leaflets of the plurality of leaflets are attached. The second commissure is attached to the frame and lies within a fourth cell axis that is substantially parallel to the longitudinal axis. The first cell axis is positioned circumferentially between the third cell axis and the fourth cell axis.

In aspects, a third separating distance circumferentially separates the first cell axis from the fourth cell axis. The first separating distance is different than the third separating distance.

In aspects, the second separating distance is different than the third separating distance.

In aspects, a first paddle is attached to the outflow end of the transcatheter heart valve prosthesis. The first paddle is axially aligned with the commissure such that the first paddle lies within the third cell axis.

In aspects, a second paddle is attached to the outflow end of the transcatheter heart valve prosthesis and is spaced circumferentially apart from the first paddle. The second paddle is axially misaligned from the second commissure.

In aspects, a transcatheter heart valve prosthesis comprises an annular frame extending along a longitudinal axis between an inflow end of the transcatheter heart valve prosthesis and an outflow end of the transcatheter heart valve prosthesis. The annular frame comprises a plurality of struts and is configured to be adjustable between a radially-collapsed position and a radially-expanded position. The plurality of struts define a first plurality of cells arranged along a first cell axis that is substantially parallel to the longitudinal axis. The first plurality of cells comprise a first cell at a first axial location and a third cell at a second axial location spaced apart from the first axial location. The first cell comprises a first shape and a first area, and the third cell comprises a third shape and a third area. One or more of the first shape is different than the third shape or the first area is different than the third area. The first cell is configured to be aligned with a first ostia such that a first radial axis intersects the longitudinal axis and passes through the first cell and the first ostia. A second plurality of cells is arranged along a second cell axis that is substantially parallel to the longitudinal axis. The second plurality of cells is spaced circumferentially apart from the first plurality of cells. The second plurality of cells comprises a second cell at the first axial location and a fourth cell at the second axial location. The second cell comprises a second shape and a second area, and the fourth cell comprises a fourth shape and a fourth area. One or more of the second shape is different than the fourth shape or the second area is different than the fourth area. A prosthetic valve is attached to the frame and comprises one or more leaflets. The prosthetic valve comprises a first edge segment and a second edge segment spaced apart and attached to the frame at the first axial location. The first cell is positioned circumferentially between the first edge segment and the second edge segment such that a first distance circumferentially separates the first cell from the first edge segment at the first axial location and a second distance circumferentially separates the first cell from the second edge segment at the first axial location. The first distance is different than the second distance.

In aspects, the second cell is configured to be aligned with a second ostia such that a second radial axis intersects the longitudinal axis and passes through the second cell and the second ostia.

In aspects, the first shape is substantially identical to the second shape and the first area is substantially identical to the second area.

In aspects, one or more of the first shape is different than the fourth shape or the first area is different than the fourth area.

In aspects, the first edge segment is circumferentially aligned with a first strut of the plurality of struts at the first axial location. The first strut lies between two cells at the first axial location.

In aspects, the second edge segment is circumferentially misaligned with and offset from the plurality of struts at the first axial location.

In aspects, methods of rotationally aligning a transcatheter heart valve prosthesis within a native heart valve. Methods comprise determining a position of a first ostia and a second ostia within the native heart valve. Methods comprise loading the transcatheter heart valve prosthesis in a first rotational orientation onto a delivery assembly. The first rotational orientation is based on the position of the first ostia and the second ostia. Methods comprise percutaneously delivering the transcatheter heart valve prosthesis to the native heart valve such that a first cell defined by one or more struts of the transcatheter heart valve prosthesis is aligned with the first ostia and a first unimpeded path extends from the first ostia and through the first cell, and a second cell defined by one or more of the struts is aligned with the second ostia and a second unimpeded path extends from the second ostia and through the second cell.

In aspects, after determining the position and before loading the transcatheter heart valve prosthesis, methods further comprise selecting the transcatheter heart valve prosthesis based on one or more of a first cell distance circumferentially separating the first cell from the second cell, a second cell distance circumferentially separating the second cell from a third cell, or a third cell distance circumferentially separating the first cell from the third cell.

In aspects, selecting the transcatheter heart valve prosthesis further comprises altering a frame of the transcatheter heart valve prosthesis to adjust one or more of the first separating distance, the second separating distance, or the third separating distance.

In aspects, selecting the transcatheter heart valve prosthesis further comprises altering a position of a prosthetic valve attached to the frame.

In aspects, selecting the transcatheter heart valve prosthesis further comprises altering a cell size of one or more of the first cell, the second cell, or the third cell to match an ostia size of one or more of the first ostia or the second ostia.

In aspects, percutaneously delivering the transcatheter heart valve prosthesis comprises positioning the first ostia circumferentially between two commissures and circumferentially centered relative to the two commissures.

In aspects, percutaneously delivering the transcatheter heart valve prosthesis comprises positioning the second ostia circumferentially between two commissures and circumferentially offset relative to the two commissures.

In aspects, the first unimpeded path and the second unimpeded path do not intersect any of the one or more struts.

Additional features and advantages of the aspects disclosed herein will be set forth in the detailed description that follows, and in part will be clear to those skilled in the art from that description or recognized by practicing the aspects described herein, including the detailed description which follows, the claims, as well as the appended drawings. It is to be understood that both the foregoing general description and the following detailed description present aspects intended to provide an overview or framework for understanding the nature and character of the aspects disclosed herein. The accompanying drawings are included to provide further understanding and are incorporated into and constitute a part of this specification. The drawings illustrate various aspects of the disclosure, and together with the description explain the principles and operations thereof.

### BRIEF DESCRIPTION OF THE DRAWINGS

These and other features, aspects and advantages are better understood when the following detailed description is read with reference to the accompanying drawings, in which:

FIG. 1 schematically illustrates example aspects of a transcatheter heart valve prosthesis in accordance with aspects of the disclosure;

FIG. 2 illustrates a top-down view of the transcatheter heart valve prosthesis in accordance with aspects of the disclosure;

FIG. 3 illustrates a side view of a delivery assembly for delivering the transcatheter heart valve prosthesis in accordance with aspects of the disclosure;

FIG. 4 illustrates a side view of the delivery assembly for delivering the transcatheter heart valve prosthesis in accordance with aspects of the disclosure;

FIG. 5 illustrates an introducer sheath in accordance with aspects of the disclosure;

FIG. 6 illustrates an introducer sheath in accordance with aspects of the disclosure;

FIG. 7 schematically illustrates a side view of the transcatheter heart valve prosthesis positioned at a treatment site in accordance with aspects of the disclosure;

FIG. 8 illustrates a top-down view of the treatment site in accordance with aspects of the disclosure;

FIG. 9 illustrates a top-down view similar to FIG. 8 with the heart valve prosthesis deployed at the treatment site in accordance with aspects of the disclosure;

FIG. 10 illustrates a flat, expanded, panoramic view of the heart valve prosthesis in accordance with aspects of the disclosure;

FIG. 11 illustrates additional aspects of the heart valve prosthesis similar to FIG. 10 in accordance with aspects of the disclosure;

FIG. 12 illustrates a spindle comprising pockets for receiving paddles of the heart valve prosthesis in accordance with aspects of the disclosure;

FIG. 13 illustrates a side view of the heart valve prosthesis relative to an ostia in accordance with aspects of the disclosure; and

FIG. 14 illustrates additional aspects of the heart valve prosthesis in accordance with aspects of the disclosure.

### DETAILED DESCRIPTION

Aspects will now be described more fully hereinafter with reference to the accompanying drawings in which example aspects are shown. Whenever possible, the same reference numerals are used throughout the drawings to refer to the same or like parts. However, this disclosure may be embodied in many different forms and should not be construed as limited to the aspects set forth herein.

As used herein, the term “about” means that amounts, sizes, formulations, parameters, and other quantities and characteristics are not, and need not be, exact, but may be approximate and/or larger or smaller, as desired, reflecting tolerances, conversion factors, rounding off, measurement error and the like, and other factors known to those of skill in the art.

Ranges can be expressed herein as from “about” one value, and/or to “about” another value. When such a range is expressed, aspects include from the one value to the other value. Similarly, when values are expressed as approximations by use of the antecedent “about,” it will be understood that the value forms another aspect. It will be further understood that the endpoints of each of the ranges are significant both in relation to the other endpoint, and independently of the other endpoint.

Directional terms as used herein—for example up, down, right, left, front, back, top, bottom, upper, lower, etc.—are made only with reference to the figures as drawn and are not intended to imply absolute orientation.

Unless otherwise expressly stated, it is in no way intended that any methods set forth herein be construed as requiring that its steps be performed in a specific order, nor that with any apparatus, specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred in any respect. This holds for any possible non-express basis for interpretation, including matters of logic relative to arrangement of steps, operational flow, order of components, or orientation of components; plain meaning derived from grammatical organization or punctuation, and; the number or type of aspects described in the specification.

As used herein, the singular forms “a,” “an,” and “the” include plural references unless the context clearly dictates otherwise. Thus, for example, reference to “a” component includes aspects having two or more such components, unless the context clearly indicates otherwise.

The word “exemplary,” “example,” or various forms thereof are used herein to mean serving as an example, instance, or illustration. Any aspect or design described herein as “exemplary” or as an “example” should not be construed as preferred or advantageous over other aspects or designs. Furthermore, examples are provided solely for purposes of clarity and understanding and are not meant to limit or restrict the disclosed subject matter or relevant portions of this disclosure in any manner. It can be appreciated that a myriad of additional or alternate examples of varying scope could have been presented but have been omitted for purposes of brevity.

As used herein, the terms “comprising,” “including,” and variations thereof shall be construed as synonymous and open-ended, unless otherwise indicated. A list of elements following the transitional phrases comprising or including is a non-exclusive list, such that elements in addition to those specifically recited in the list may also be present.

The terms “substantial,” “substantially,” and variations thereof as used herein are intended to represent that a described feature is equal or approximately equal to a value or description. For example, a “substantially planar” surface is intended to denote a surface that is planar or approximately planar. Moreover, “substantially” is intended to denote that two values are equal or approximately equal. The term “substantially” may denote values within about 10% of each other, for example, within about 5% of each other, or within about 2% of each other.

Modifications may be made to the instant disclosure without departing from the scope or spirit of the claimed subject matter. Unless specified otherwise, “first,” “second,” or the like are not intended to imply a temporal aspect, a spatial aspect, an ordering, etc. Rather, such terms are merely used as identifiers, names, etc. for features, elements, items, etc. For example, a first end and a second end generally correspond to end A and end B or two different ends.

Unless otherwise indicated, the terms “distal” and “proximal” are used in the following description with respect to a position or direction relative to the treating clinician. “Distal” and “distally” are positions distant from or in a direction away from the clinician, and “proximal” and “proximally” are positions near or in a direction toward the clinician. In addition, the term “self-expanding” may be used in the following description with reference to one or more valve or stent structures of the prostheses hereof and is intended to convey that the structures are shaped or formed from a material that can be provided with a mechanical memory to return the structure from a compressed or constricted delivery configuration to an expanded deployed configuration or vice versa. Non-exhaustive exemplary self-expanding materials include stainless steel, a pseudo-elastic metal such as a nickel titanium alloy or nitinol, various polymers, or a so-called super alloy, which may have a base metal of nickel, cobalt, chromium, or other metal. Mechanical memory may be imparted to a wire or stent structure by thermal treatment to achieve a spring temper in stainless steel, for example, or to set a shape memory in a susceptible metal alloy, such as nitinol. Various polymers that can be made to have shape memory characteristics may also be suitable for use in aspects hereof to include polymers such as polynorborene, trans-polyisoprene, styrene-butadiene, and polyurethane. As well poly L-D lactic copolymer, oligo caprylactone copolymer and poly cyclo-octine can be used separately or in conjunction with other shape memory polymers.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses generally include a frame or stent and a prosthetic valve mounted within the frame. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

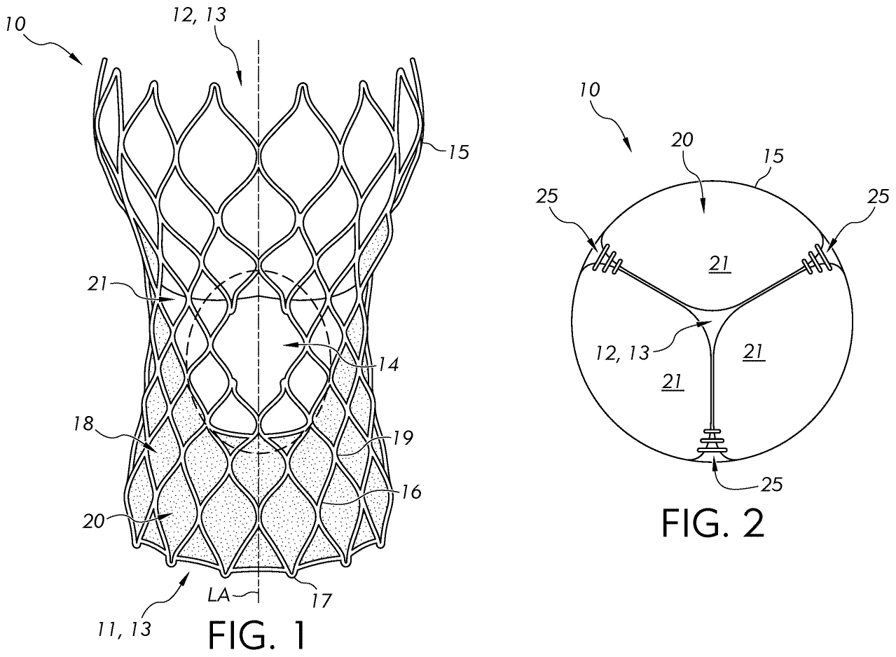

FIGS. 1 and 2 illustrate an example transcatheter heart valve prosthesis 10. The delivery assemblies described herein may be used with the transcatheter heart valve prosthesis 10 and/or other transcatheter heart valve prostheses. The transcatheter heart valve prosthesis 10 is illustrated to facilitate description of the disclosure. The following description of the transcatheter heart valve prosthesis 10 is merely exemplary in nature and is not intended to limit the invention or the application and uses of the invention.

FIGS. 1 and 2 illustrate a side view and a top (outflow end) view, respectively, of the transcatheter heart valve prosthesis 10. The transcatheter heart valve prosthesis 10 includes a radially-expandable frame or stent 15 and a prosthetic valve 20. The frame 15 of the transcatheter heart valve prosthesis 10 supports the prosthetic valve 20 within an interior of the frame 15, for example, with the prosthetic valve 20 attached to the frame 15. In the example transcatheter heart valve prosthesis 10 shown in FIGS. 1 and 2, the frame 15 is self-expandable. However, this is not meant to be limiting, and the frame 15 can be balloon-expandable or mechanically expandable in other embodiments. The frame 15 comprises a plurality of struts 16 such that the frame 15 is adjustable between a radially-collapsed position and a radially-expanded position.

The prosthetic valve 20 includes at least one leaflet 21 disposed within and secured to the frame 15. In the embodiment shown in FIGS. 1 and 2, the prosthetic valve 20 includes exactly three leaflets 21, as shown in FIG. 2. However, this is not meant to be limiting, as the prosthetic valve 20 may include more or fewer leaflets 21. The valve leaflets 21 open and close to regulate flow through the transcatheter heart valve prosthesis 10.

As shown in FIG. 1, the transcatheter heart valve prosthesis 10 includes an inflow end 11 and an outflow end 12. The prosthetic leaflets 21 are attached to the frame 15 at commissures 25 such that when pressure at the inflow end 11 exceeds pressure at the outflow end 12, the prosthetic leaflets 21 open to allow blood flow through the heart valve prosthesis 10 from the inflow end 11 to the outflow end 12. When the pressure at the outflow end 12 exceeds pressure at the inflow end 11, the prosthetic leaflets 21 close to prevent blood flow from the outflow end 12 to the inflow end 11. Accordingly, the at least one leaflet (e.g., the prosthetic leaflets 21) can be attached to the plurality of struts 16, for example, by being directly attached to the plurality of struts 16 at the commissures 25, or by being indirectly attached to the plurality of struts 16, for example, by being attached to a skirt, a commissure bracket, or other structure (e.g., mechanical actuator) that is attached to the plurality of struts 16.

The frame 15 of the transcatheter heart valve prosthesis 10 further includes a plurality of struts 16 that are arranged to form a plurality of openings or cells 18 arranged circumferentially around a longitudinal axis LA of the transcatheter heart valve prosthesis 10 and longitudinally to form a tubular structure defining a central lumen 13 of the transcatheter heart valve prosthesis 10. For example, the frame 15 can extend along the longitudinal axis LA between the inflow end 11 and the outflow end 12. The frame 15 is configured to secure the prosthetic valve 20 within the central lumen 13 of the frame 15 and to secure the transcatheter heart valve prosthesis 10 in place in the vasculature of the patient. The struts 16 are defined herein as the elongated wire segments of the frame 15. Struts 16 come together to form crowns 17 or nodes 19, as can be seen in FIG. 1. The frame 15 of the heart valve prosthesis 10 includes a plurality of cells 18 defined as the spaces between the plurality of crowns 17, the plurality of nodes 19, and the plurality of struts 16. The frame 15, and, thus, the plurality of struts 16, can be adjustable between a radially-collapsed position and a radially-expanded position.

In the example embodiment shown in FIG. 1, the plurality of cells 18 may be diamond-shaped. In the example embodiment shown, the plurality of cells include a plurality of first cells 18 and access cells 14. In particular, the access cells are larger than the first cells 18 and can provide access to one or more coronary arteries when the transcatheter heart valve prosthesis 10 is implanted in the patient. In the embodiment shown, there are exactly three access cells 14. However, this is not meant to be limiting, as the frame 15 of the transcatheter heart valve prosthesis 10 can include more, fewer, or no access cells 14. The access cells 14 each have an enlarged area relative or compared to the first cells 18, as can be seen in FIG. 1. Further, the access cells 14 may be located in other locations than the locations shown in FIG. 1. Although not shown, in some embodiments the transcatheter heart valve prosthesis 10 may include an outer skirt extending circumferentially around an outer circumference of the stent 15 at or near the inflow end 11 to prevent paravalvular leakage of blood around the outside of the transcatheter heart valve prosthesis 10 once implanted in the patient.

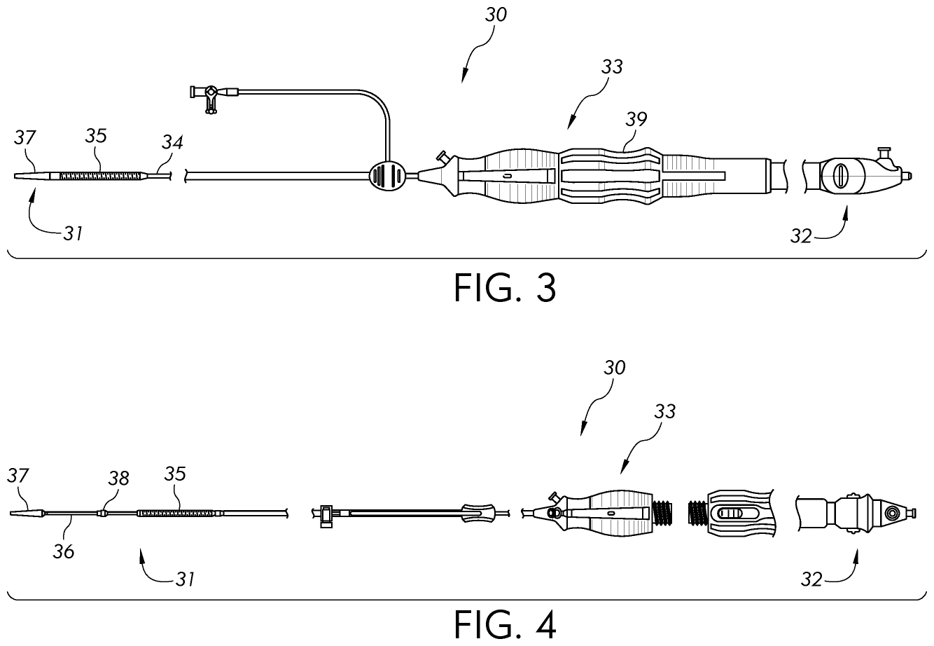

FIGS. 3 and 4 show schematically side views of a delivery assembly 30 for delivering and deploying a transcatheter heart valve prosthesis (e.g., transcatheter heart valve prosthesis 10) according to embodiments hereof. One skilled in the art will realize that FIGS. 3 and 4 illustrate one example of a delivery assembly 30 and that components illustrated in FIGS. 3 and 4 may be removed and/or additional components may be added. The delivery assembly 30 includes a distal end 31, a proximal end 32, and a handle 33. The handle 33 enables a physician to manipulate a distal portion of the delivery assembly 30 and includes actuators for moving parts of the delivery assembly 30 relative to other parts. In the delivery assembly 30, an outer shaft 34 is coupled to an actuator 39 of the handle 33 for moving the outer shaft 34 relative to an inner shaft 36.

A distal portion of the outer shaft 34, referred to as a capsule 35, is configured to surround a transcatheter heart valve prosthesis (e.g., transcatheter heart valve prosthesis 10) during delivery to the treatment site (e.g., a native heart valve) and is retracted from the transcatheter heart valve prosthesis to expose the transcatheter heart valve prosthesis such that it self-expands. The inner shaft 36 is coupled to the handle 33 and movement of the handle 33 translates to movement of the inner shaft 36 and a distal tip or nosecone 37 coupled to a distal end of the inner shaft 36. The inner shaft 36 and distal tip or nosecone 37 may also be translated relative to the outer shaft 34 and the handle 33 via a tip retractor. In the embodiment shown, the inner shaft 36 includes a retainer or spindle 38 for receiving the paddles of the transcatheter heart valve prosthesis 10.

When the actuator 39 is actuated, the actuator 39 moves the outer shaft 34 and the capsule 35 relative to the inner shaft 36, as shown in FIG. 4. As known to those skilled in the art, when the delivery assembly 30 is in position such that the transcatheter heart valve prosthesis 10 is at the desired position at the treatment site in the patient's vasculature, the actuator 39 is actuated to move the capsule 35 relative to the inner shaft 36 and the transcatheter heart valve prosthesis 10 disposed between the inner shaft 36 and the capsule 35, thereby enabling the transcatheter heart valve prosthesis 10 to deploy via self-expansion at the treatment site and release from the spindle 38, as shown in FIG. 4 (without showing the transcatheter heart valve prosthesis 10).

Minimally invasive percutaneous interventional procedures, including endovascular procedures, require access to the venous or arterial system. In general, it is desirable to make the smallest incision point with the shortest tissue contact time when entering the body. Small incisions and short tissue contact time generally lead to improved patient outcomes, less complications, and less trauma to the vessels or organs being accessed, as well as less trauma to the skin and tissue through which the access point is created. Access is required for various medical procedures that deliver or implant structural elements (such as heart valves, heart valve repair devices, occluders, grafts, electrical stimulators, leads, etc.) percutaneously. Some procedures employ relatively large devices that require relatively large sheaths to deliver the devices to the intended site within the body. With such procedures, access site trauma can occur, often resulting in vessel damage, excessive bleeding, increased case time, increased risk of infection, and increased hospitalization time. To reduce access trauma, physicians try to use the smallest devices possible and place the smallest sheath size. This can be problematic, however, if during the procedure the physician discovers a larger device is needed. This leads to a need to upsize the sheath, which is a lengthy procedure and leads to increased risk to the patient. Expandable sheaths can be expanded within the body and thus do not require removal to upsize.

Expandable sheath designs may be regionally or locally expansive to selectively and temporarily expand when the device is passing through a region of the sheath and to retract or recover when the device is not passing or has already passed through the sheath. Embodiments disclosed herein may be employed with an expandable introducer sheath that may solve these and other issues that contribute to vascular trauma. The expandable introducer sheath disclosed herein is described with respect to percutaneous access for transcatheter heart valve repair or replacement, and it should be understood that one or more features of the expandable introducer sheath may be employed alone or in combination for other medical procedures requiring percutaneous access, including but not limited to placement of stents, angioplasty, removal of arterial or venous calcification, and pre-dilatation or post-dilatation.

Various embodiments disclosed herein may include an introducer sheath that has a selectively expandable diameter to allow for the passage of a relatively larger device therethrough and further is configured to return to its original diameter upon passage of the device. The various embodiments may reduce damage to surrounding tissues by reducing contact with those tissues and by eliminating the need to exchange sheaths of different sizes. As a result, in comparison to known sheaths, these embodiments can reduce procedure time, vascular trauma, bleeding, and the resulting risk of infection and other complications. However, it should be understood that the present disclosure is not limited for use with an expandable introducer sheath. Rather, one or more features of the present disclosure can be employed either alone or in combination without an introducer sheath, with a non-expandable introducer sheath, or with an expandable introducer sheath. Likewise, if employed, the introducer sheath may be an integrated introducer sheath (e.g., an introducer sheath integrated with a delivery assembly) or a non-integrated introducer sheath (e.g., an introducer sheath separate from the delivery assembly but provided for use with the delivery assembly).

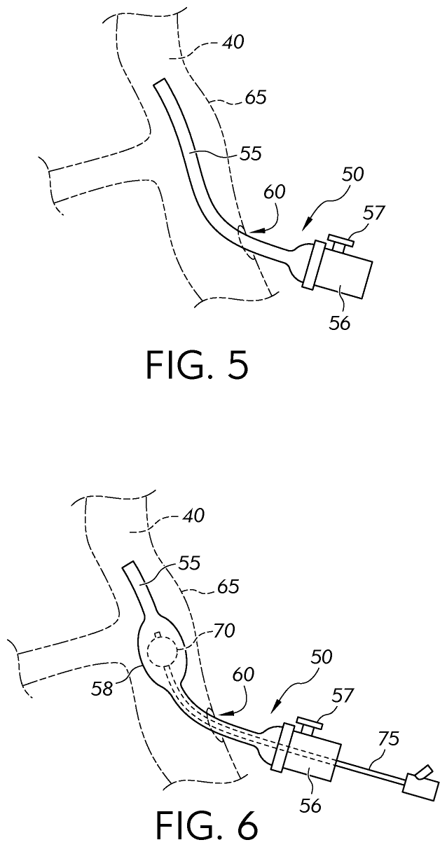

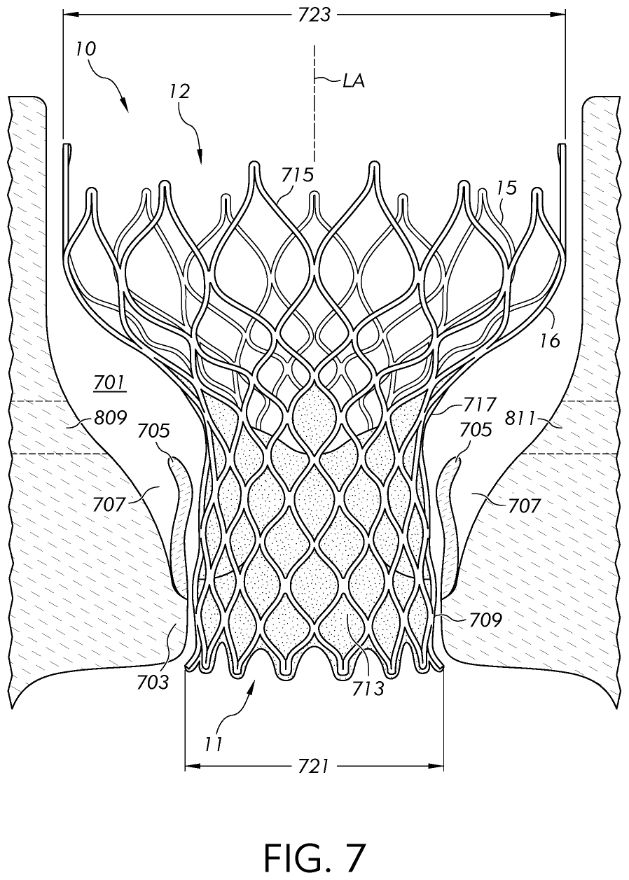

FIGS. 5 and 6 depict one embodiment of an introducer sheath 50 positioned through an incision 60 in the skin 65 of a patient and into a vessel 40 of a patient. The sheath 50 has a tubular shaft 55 and a proximal hub 56 with a hemostatic seal and a luer lock 57. FIG. 5 shows the sheath 50 positioned in the vessel 40 in its normal, unexpanded state, while FIG. 6 shows the sheath 50 positioned in the vessel 40 with a delivery device 75 delivering another device 70 that is being advanced through the sheath 50 such that the tubular shaft 55 expands or deforms at the location where the device 70 is passing through. The shaft 55 expands at expanded region 58 when the device 70 passes through and then retracts or recovers to its original diameter after the device 70 moves past or is removed from the shaft 55. Thus, the tubular shaft 55 is configured to be expandable and retractable.

In certain embodiments, the expandability of the shaft 55 (and any shaft described according to any embodiment set forth herein) is achieved via the elasticity of the shaft 55, which can result in the shaft 55 being either self-expandable or self-expanding or mechanically expandable or mechanically expanding. For purposes of this application, self-expandable means that the shaft 55 is configured to expand to a predetermined or nominal diameter automatically (without any type of actuation, mechanical or otherwise). Further, for purposes of this application, mechanically expandable means that the shaft 55 is configured to expand when a positionable medical device is positioned through the shaft 55. That is, the device itself that is being passed through the shaft 55 causes the expansion of the shaft 55, as depicted in FIG. 6. Alternatively, the expandable characteristics of the shaft 55 can be caused by something other than elasticity.

After passage of the device, the shaft 55 is configured to be contractable, retractable, or recoverable to its original, unexpanded state as depicted in FIG. 5. The retractability can be, in certain embodiments, achieved by the elasticity of the shaft 55, which can result in the shaft 55 being either self-retractable or self-retracting, self-recoverable, or self-contractable, or mechanically retractable or mechanically retracting, mechanically recoverable, or mechanically contractable. For purposes of this application, self-retractable means that the shaft 55 is configured to retract to a predetermined or nominal diameter automatically (without any type of actuation, mechanical or otherwise). Further, for purposes of this application, mechanically retractable means that the shaft 55 is configured to retract when a device or component is used to cause the shaft 55 to retract or recover. Alternatively, the retractable characteristics of the shaft 55 can be caused by something other than elasticity.

For purposes of this application, any device that can be positioned through an introducer sheath according to any embodiment disclosed or contemplated herein can be referred to as a positionable medical device or insertable medical device. Such devices include guidewires, dilators, delivery devices (for delivery and/or placement of structural elements such as heart valves, heart valve repair devices, occluders, grafts, electrical stimulators, leads, etc.), guide catheters, guiding sheaths, diagnostic catheters, stent delivery systems, balloon catheters, and other known vascular devices. Other devices can include non-vascular devices such as scopes and other common surgical instruments. Further, the introducer sheath is configured to receive tissues or organs. Thus, as one non-limiting example, the introducer sheath 50 is described as being an expandable introducer sheath 50 for introduction of a delivery assembly 30 including a transcatheter heart valve prosthesis 10.

FIG. 7 illustrates the heart valve prosthesis 10 at a treatment site 701 within a patient's vasculature. In aspects, the treatment site 701 can comprise a location of a native aortic annulus (hereinafter “annulus”) 703 of a native heart valve, for example, the annulus of a patient's left ventricle. The treatment site 701 can comprise one or more native valve leaflets 705 and corresponding native sinuses 707. Although disclosed with respect to placement of the heart valve prosthesis 10 within a native anatomy of the patient, in aspects, the heart valve prosthesis 10 can be implanted within a previously implanted prosthetic valve (e.g., a surgical or transcatheter index valve) to facilitate a valve-in-valve (e.g., TAV-in-SAV or TAV-in-TAV) procedure, without departing from the scope of the disclosure. In aspects, paravalvular leakage can occur when blood travels through a gap 709 around the outside of the transcatheter heart valve prosthesis 10, with the gap 709 formed between the transcatheter heart valve prosthesis 10 and the annulus 703. To avoid paravalvular leakage, the heart valve prosthesis 10 can be radially expanded such that an outer radial surface of the heart valve prosthesis 10 can contact the annulus 703 and/or the native valve leaflets 705, thus reducing or eliminating the gap 709 and causing the blood to flow through the central lumen 13 of the heart valve prosthesis 10. The frame 15 of the heart valve prosthesis 10 can comprise an asymmetric hourglass shape with a first section 713 at the inflow end 11, a second section 715 at the outflow end 12, and a waist section 717 positioned between the first section 713 and the second section 715. In aspects, the first section 713 can comprise a first diameter 721 and the second section 715 can comprise a second diameter 723, with the second diameter 723 greater than the first diameter 721. Additionally, as discussed above, in some embodiments the transcatheter heart valve prosthesis 10 may include an outer skirt extending circumferentially around an outer circumference of the frame 15 at or near the inflow end 11 to prevent paravalvular leakage of blood around the outside of the transcatheter heart valve prosthesis 10 once implanted in the patient. Thus, features of the disclosure may be employed alone or in combination with a heart valve prosthesis 10 having an outer skirt or other external sealing member (not shown) or a heart valve prosthesis 10 having no outer skirt.

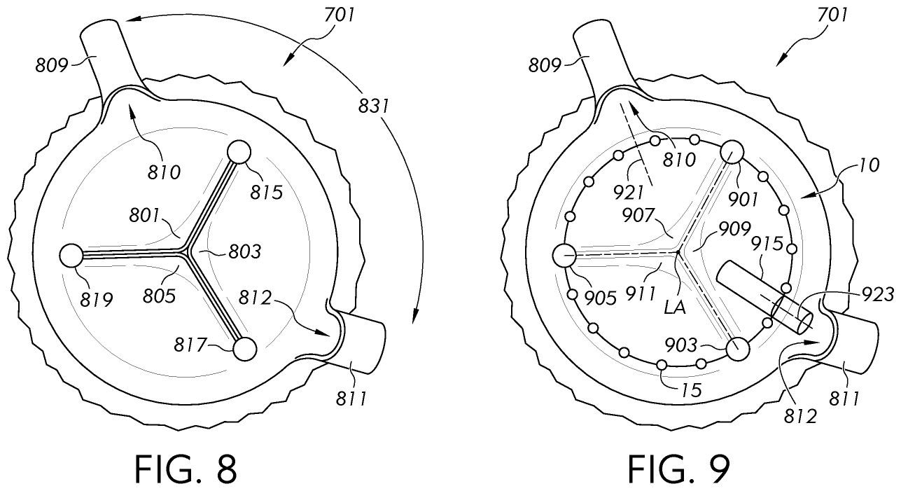

FIG. 8 illustrates a top-down view of the treatment site 701 without the heart valve prosthesis 10, wherein the treatment site 701 comprises an aortic valve in a view from the aorta. The native aortic valve can comprise three leaflets or cusps, for example, a first native leaflet 801, a second native leaflet 803, and a third native leaflet 805. The treatment site 701 comprises a first coronary artery 809 (e.g., right coronary artery), which comprises a first ostia 810 or opening in the sinus of Valsalva, and a second coronary artery 811, which comprises a second ostia 812 or opening in the sinus of Valsalva. The native leaflets 801, 803, 805 can be joined at native commissures 815, 817, 819. For example, the first native commissure 815 is where the first native leaflet 801 and the second native leaflet 803 are joined. The second native commissure 817 is where the second native leaflet 803 and the third native leaflet 805 are joined. The third native commissure 819 is where the first native leaflet 801 and the third native leaflet 805 are joined. It will be appreciated that the native commissures 815, 817, 819 may not be in the same location for all patients, and, in aspects, a patient-specific variation of 10-20 degrees is possible. In this way, the native commissures 815, 817, 819 may not be exactly 120 degrees apart. Rather, in aspects, the first native commissure 815 and the second native commissure 817 can be closer, for example, about 108 degrees apart. Likewise, the location of the ostia 810, 812 may vary approximately 15-20 degrees depending on patient anatomy.

FIG. 9 illustrates the treatment site 701 of FIG. 8 after the delivery and deployment of the valve prosthesis 10 at the treatment site 701. For example, the valve prosthesis 10 can comprise a first commissure 901, a second commissure 903, and a third commissure 905 attached to the frame 15, with the commissures 901, 903, 905 substantially identical to the commissures 25 of FIG. 2. In aspects, the valve prosthesis 10 can be positioned such that the first commissure 901 is placed at a substantially identical position as the first native commissure 815, the second commissure 903 is placed at a substantially identical position as the second native commissure 817, and the third commissure 905 is placed at a substantially identical position as the third native commissure 819. The valve prosthesis 10 can comprise one or more leaflets, for example, a first leaflet 907, a second leaflet 909, and a third leaflet 911, with the first leaflet 907 placed at a substantially identical position as the first native leaflet 801, the second leaflet 909 placed at a substantially identical position as the second native leaflet 803, and the third leaflet 911 placed at a substantially identical position as the third native leaflet 805. In this way, the first commissure 901 is where a first pair of the leaflets 907, 909 are attached, with the first commissure 901 attached to the frame 15. The second commissure 903 is where a second pair of the leaflets 909, 911 are attached, with the second commissure 903 attached to the frame 15. The third commissure 905 is where a third pair of the leaflets 907, 911 are attached, with the third commissure 905 attached to the frame 15.

In aspects, subsequent to the delivery and deployment in situ of the valve prosthesis 10 at the treatment site 701, access to one or both of the first coronary artery 809 or the second coronary artery 811 through the first ostia 810 and/or the second ostia 812 may be necessary. For example, a physician may need to access the first coronary artery 809 and/or the second coronary artery 811 with a coronary guide catheter 915. To limit the likelihood of obstructing the first coronary artery 809 and/or the second coronary artery 811 and, thus, blocking the coronary guide catheter 915 from passing through the first ostia 810 and/or the second ostia 812, the valve prosthesis 10 can be positioned such that the struts 16 of the frame 15, the commissures 901, 903, 905, and the prosthetic valve 20 do not obstruct the first ostia 810 and the second ostia 812. For example, a first radial axis 921 can intersect the longitudinal axis LA and pass through the first ostia 810 and the first coronary artery 809. The first radial axis 921 may be perpendicular to the longitudinal axis LA, and the first radial axis 921 may not intersect or pass through the struts 16 of the frame 15, the commissures 901, 903, 905, or the prosthetic valve 20. Likewise, a second radial axis 923 can intersect the longitudinal axis LA and pass through the second ostia 812 and the second coronary artery 811. The second radial axis 923 may be perpendicular to the longitudinal axis LA, and the second radial axis 923 may not intersect or pass through the struts 16 of the frame 15, the commissures 901, 903, 905, or the prosthetic valve 20. Accordingly, in this way, the valve prosthesis 10 can be positioned to rotationally align the prosthetic commissures and leaflets with the native commissures and leaflets, while also not blocking or obstructing the ostia 810, 812, thus allowing subsequent access to the coronary arteries 809, 811 via the coronary guide catheter 915.

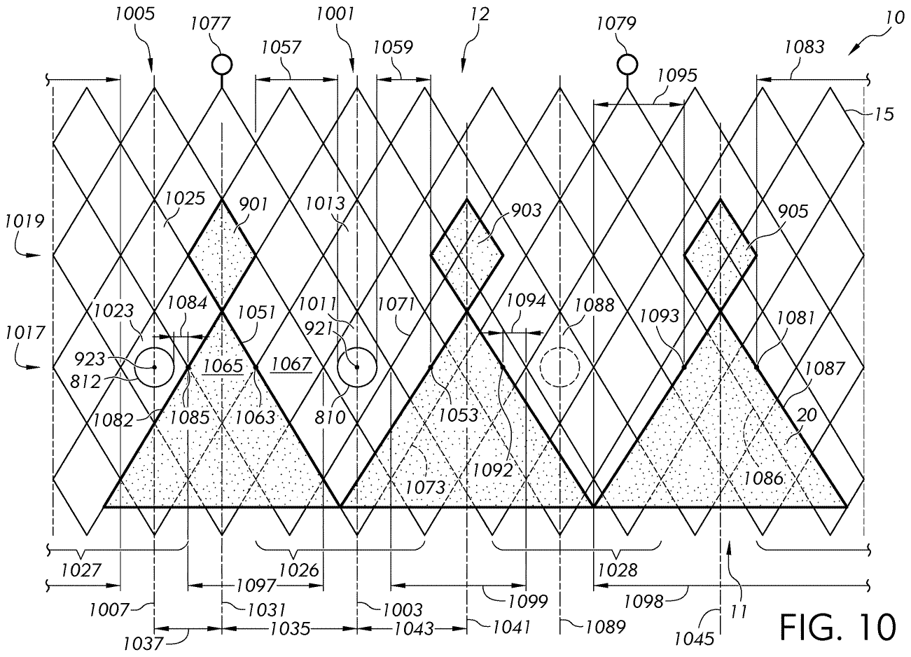

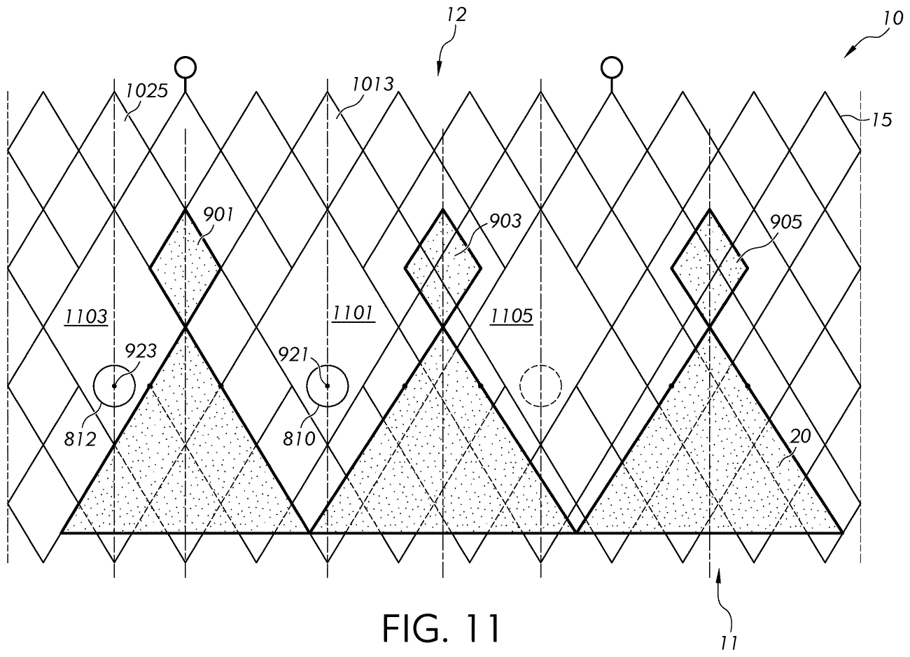

FIG. 10 illustrates a flat, expanded, panoramic view of the valve prosthesis 10 as viewed from a radial perspective of the longitudinal axis LA. It will be appreciated that, in operation, the valve prosthesis 10 comprises the shape illustrated in FIGS. 7 and 9, such that the right edge of the valve prosthesis 10 in FIG. 10 is attached to the left edge of the valve prosthesis 10 in FIG. 10. It will further be appreciated that, as described herein, the valve prosthesis 10 can be rotated depending on the position of the ostia 810, 812, such that the valve prosthesis 10 is not limited to the rotational position (e.g., relative to the ostia 810, 812) illustrated in FIG. 10. The plurality of struts 16 can define one or more plurality of cells around a circumference and along a length of the valve prosthesis 10, with each cell of the plurality of cells bounded by one or more struts 16. In aspects, the plurality of struts 16 can define a first plurality of cells 1001 arranged along a first cell axis 1003 that is substantially parallel to the longitudinal axis LA, and a second plurality of cells 1005 arranged along a second cell axis 1007 that is substantially parallel to the longitudinal axis LA and the first cell axis 1003.

The first plurality of cells 1001 comprises cells that are spaced apart and arranged along the first cell axis 1003, for example, with the first plurality of cells 1001 comprising a first cell 1011, a third cell 1013, etc. The first cell 1011 is at a first axial location 1017 and the third cell 1013 is at a second axial location 1019. The axial locations 1017, 1019 are spaced apart along the longitudinal axis LA, with the first axial location 1017 closer to the inflow end 11 than the second axial location 1019, and the second axial location 1019 closer to the outflow end 12 than the first axial location 1017. In aspects, the first cell 1011 can be aligned with the first ostia 810 such that the first radial axis 921 intersects the longitudinal axis LA and passes through the first cell 1011 and the first ostia 810. In this way, a first unimpeded path can extend from the first ostia 810 and through the first cell 1011 along the first radial axis 921. The first unimpeded path comprises, for example, a cross-sectional shape and size that matches a cross-sectional shape and size of the first ostia 810 and the first coronary artery 809, such that the first unimpeded path does not intersect any of the struts 16, the commissures 901, 903, 905, or the prosthetic valve 20. In this way, the first ostia 810 is not blocked by the valve prosthesis 10 such that access to the first ostia 810 and the first coronary artery 809 is provided to the coronary guide catheter 915 through the first cell 1011 along the first unimpeded path.

The second plurality of cells 1005 comprises cells that are spaced apart and arranged along the second cell axis 1007, for example, with the second plurality of cells 1005 comprising a second cell 1023, a fourth cell 1025, etc. The second cell 1023 is at the first axial location 1017 and the fourth cell 1025 is at the second axial location 1019. The second plurality of cells 1005, and, thus, the second cell axis 1007, may be spaced circumferentially apart from the first plurality of cells 1001 and, thus, the first cell axis 1003. As used herein, the terms “circumferential,” “circumferentially,” “circumference,” or variations thereof can be understood to refer to the distance or path around the perimeter of the frame 15 due to the curved shape of the valve prosthesis 10. In this way, for example, a circumferential distance between two points that are circumferentially offset by 180 degrees is the distance around the perimeter of the frame 15 between the two points and not the diameter or linear distance through the frame 15. In aspects, the second cell 1023 can be aligned with the second ostia 812 such that the second radial axis 923 intersects the longitudinal axis LA and passes through the second cell 1023 and the second ostia 812. In this way, a second unimpeded path can extend from the second ostia 812 and through the second cell 1023 along the second radial axis 923. The second unimpeded path comprises, for example, a cross-sectional shape and size that matches a cross-sectional shape and size of the second ostia 812 and the second coronary artery 811, such that the second unimpeded path does not intersect any of the struts 16, the commissures 901, 903, 905, or the prosthetic valve 20. In this way, the second ostia 812 is not blocked by the valve prosthesis 10 such that access to the second ostia 812 and the second coronary artery 811 is provided to the coronary guide catheter 915 through the second cell 1023 along the seconded unimpeded path.

In aspects, the first cell 1011 and/or the third cell 1013 can comprise shapes that are the same or different, and, in aspects, the second cell 1023 and/or the fourth cell 1025 can comprise shapes that are the same or different. For example, the first cell 1011 can comprise a first shape (e.g., a quadrilateral shape such as a square, rectangle, parallelogram, rhombus, etc.) and a first area and the third cell 1013 can comprise a third shape (e.g., a quadrilateral shape such as a square, rectangle, parallelogram, rhombus, etc.) and a third area. As illustrated in FIG. 9, in aspects, the first shape may be substantially identical to the third shape, and the first area may be substantially identical to the third area. In addition, the second cell 1023 can comprise a second shape (e.g., a quadrilateral shape such as a square, rectangle, parallelogram, rhombus, etc.) and a second area and the fourth cell 1025 can comprise a fourth shape (e.g., a quadrilateral shape such as a square, rectangle, parallelogram, rhombus, etc.) and a fourth area. As illustrated in FIG. 9, in aspects, the second shape may be substantially identical to the fourth shape, and the second area may be substantially identical to the fourth area. Likewise, in aspects, the first shape can be substantially identical to the second shape and the first area can be substantially identical to the second area. However, as explained relative to FIGS. 11 and 14, in aspects, some of the cells can differ in size and/or shape from other cells due, for example, to providing access to the ostia 810, 812.

The first commissure 901 can lie within a third cell axis 1031 that is substantially parallel to the longitudinal axis LA. The third cell axis 1031 can be positioned circumferentially between the first cell axis 1003 and the second cell axis 1007. In aspects, a first separating distance 1035 can circumferentially separate the first cell axis 1003 from the third cell axis 1031, such that the first cell 1011 is circumferentially offset from the first commissure 901. A second separating distance 1037 can circumferentially separate the second cell axis 1007 from the third cell axis 1031, such that the second cell 1023 is circumferentially offset from the first commissure 901. In aspects, the first separating distance 1035 is different than the second separating distance 1037, such that the first cell 1011 is spaced a circumferential distance from the first commissure 901 that is different than a circumferential distance between the first commissure 901 and the second cell 1023.

The second commissure 903 can lie within a fourth cell axis 1041 that is substantially parallel to the longitudinal axis LA. The first cell axis 1003 can be positioned circumferentially between the third cell axis 1031 and the fourth cell axis 1041. In this way, the first cell 1011 can be positioned circumferentially between the first commissure 901 and the second commissure 903. The second cell 1023 can be positioned circumferentially between the first commissure 901 and the third commissure 905. A third separating distance 1043 can circumferentially separate the first cell axis 1003 from the fourth cell axis 1041, with the first separating distance 1035 different than the third separating distance 1043. Likewise, in aspects, the second separating distance 1037 can be different than the third separating distance 1043. The third commissure 905 can lie within a fifth cell axis 1045 that is substantially parallel to the longitudinal axis LA.

The prosthetic valve 20 can comprise a first edge segment 1051 and a second edge segment 1053 that are spaced apart and attached to the frame 15 at the first axial location 1017. The first edge segment 1051 and the second edge segment 1053 (e.g., and the other edge segments described herein) can comprise, for example, edges of the prosthetic valve 20 that extend from the commissures 901, 903, 905 toward the inflow end 11 of the valve prosthesis 10. In aspects, the edge segments described herein can comprise, for example, edges of the leaflets. By being attached to the frame 15, the edge segments 1051, 1053 can be attached to struts 16 of the frame 15, for example, by sutures or other mechanical fasteners. The first edge segment 1051 can be attached to, and extend toward, the first commissure 901, and the second edge segment 1053 can be attached to, and extend toward, the second commissure 903. The first cell 1011 can be positioned circumferentially between the first edge segment 1051 and the second edge segment 1053, with zero other edge segments between the first cell 1011 and the first edge segment 1051 and zero other edge segments between the first cell 1011 and the second edge segment 1053.

In aspects, a first distance 1057 can circumferentially separate the first cell 1011 from the first edge segment 1051 at the first axial location 1017, and a second distance 1059 can circumferentially separate the first cell 1011 from the second edge segment 1053 at the first axial location 1017. The first distance 1057 may be different than the second distance 1059, for example, with the first distance 1057 greater than the second distance 1059. In this way, the first cell 1011 is circumferentially offset from, and not at a midpoint between, the first edge segment 1051 and the second edge segment 1053. Likewise, the first cell 1011 is circumferentially offset from, and not at a midpoint between, the commissure 901 and the second commissure 903. For example, the first edge segment 1051 can be attached to, and circumferentially aligned with, a first strut 1063 at the first axial location 1017, with the first strut 1063 lying between two cells 1065, 1067 at the first axial location 1017. By being circumferentially aligned, a radial axis can extend from the longitudinal axis LA and pass through the first strut 1063 and the first edge segment 1051 at the first axial location 1017, with the first edge segment 1051 attached to, parallel with, and in contact with the first strut 1063. The second edge segment 1053 can be circumferentially misaligned with, and offset from, the struts 16 at the first axial location 1017. For example, the second edge segment 1053 can be positioned between a second strut 1071 and a third strut 1073, with the second strut 1071 and the third strut 1073 the closest struts to the second edge segment 1053 at the first axial location 1017. By being circumferentially mis-aligned, a radial axis can extend from the longitudinal axis LA and pass through the second edge segment 1053 while not passing through any struts (e.g., the second strut 1071, the third strut 1073, etc.) at the first axial location 1017, with the second edge segment 1053 spaced a distance apart from the second strut 1071 and a distance apart from the third strut 1073 at the first axial location 1017.

The valve prosthesis 10 can comprise one or more paddles that can facilitate alignment and deployment of the valve prosthesis 10. For example, in aspects, the valve prosthesis 10 can comprise a first paddle 1077 and a second paddle 1079 attached to the outflow end 12 of the frame 15. The second paddle 1079 can be spaced circumferentially apart from the first paddle 1077. In aspects, the first paddle 1077 and the second paddle 1079 can be spaced circumferentially about 170 degrees to about 190 degrees, or about 180 degrees apart. The first paddle 1077 can be axially aligned with the first commissure 901 such that the first paddle 1077 lies within the third cell axis 1031. The second paddle 1079 can be axially misaligned from the second commissure 903 and the third commissure 905, such that the second lies in a different axis than the fourth cell axis 1041 and the fifth cell axis 1045. While two paddles 1077, 1079 are illustrated in FIG. 10, in further aspects, the valve prosthesis 10 can comprise greater than two paddles, for example, three paddles, four paddles, five paddles, etc. In aspects, the paddles 1077, 1079 are radiopaque so as to be visible under fluoroscopy, with the first paddle 1077 comprising a C-shaped marker to assist with the orientation of the valve prosthesis 10 during implantation. The valve prosthesis 10 is not limited to comprising the paddles 1077, 1079 and, in addition or in the alternative, can comprise eyelets, loops, slots, or any other suitable coupling member.

The prosthetic valve 20 can comprise additional edge segments between which the second cell 1023 is positioned. For example, the prosthetic valve 20 can comprise a third edge segment 1081 and a fourth edge segment 1082 that are spaced apart and attached to the frame 15 at the first axial location 1017. By being attached to the frame 15, the edge segments 1081, 1082 can be attached to struts 16 of the frame 15, for example, by sutures or other mechanical fasteners. The third edge segment 1081 can be attached to, and extend toward, the third commissure 905, and the fourth edge segment 1082 can be attached to, and extend toward, the first commissure 901. The second cell 1023 can be positioned circumferentially between the third edge segment 1081 and the fourth edge segment 1082, with zero other edge segments between the second cell 1023 and the third edge segment 1081, and zero other edge segments between the second cell 1023 and the fourth edge segment 1082.

In aspects, a third distance 1083 can circumferentially separate the second cell 1023 from the third edge segment 1081 at the first axial location 1017, and a fourth distance 1084 can circumferentially separate the second cell 1023 from the fourth edge segment 1082 at the first axial location 1017. The third distance 1083 may be different than the fourth distance 1084, for example, with the third distance 1083 greater than the fourth distance 1084. In this way, the second cell 1023 is circumferentially offset from, and not at a midpoint between, the third edge segment 1081 and the fourth edge segment 1082. Likewise, the second cell 1023 is circumferentially offset from, and not at a midpoint between, the first commissure 901 and the third commissure 905. In aspects, the fourth edge segment 1082 can be attached to, and circumferentially aligned with, a strut 1085 at the first axial location 1017, similar to the first edge segment 1051 circumferentially aligned with the first strut 1063. The strut 1085 can lie between two cells at the first axial location 1017 such that a radial axis can extend from the longitudinal axis LA and pass through the strut 1085 and the fourth edge segment 1082. The third edge segment 1081 can be circumferentially misaligned with, and offset from, the struts 16 at the first axial location 1017. For example, the third edge segment 1081 can be positioned between struts 1086, 1087, with the struts 1086, 1087 being the closest struts to the third edge segment 1081 at the first axial location 1017. As such, the third edge segment 1081 is spaced a distance apart from each of the struts 1086, 1087 at the first axial location 1017. In this way, edge segments that are spaced a distance apart from struts at the first axial location 1017 are not in direct contact with struts at the first axial location, but, rather, can lie between neighboring adjacent struts.

The valve prosthesis 10 is not limited to comprising two cells (e.g., the first cell 1011 and the second cell 1023) that can be aligned with the ostia 810, 812. Rather, the valve prosthesis 10 comprises a fifth cell 1088 positioned at the first axial location 1017 and lying in an axis 1089 that is between the fourth cell axis 1041 and the fifth cell axis 1045. The fifth cell 1088 may be substantially identical in structure, size, and shape to the first cell 1011 and/or the second cell 1023. For example, the fifth cell 1088 can comprise a fifth shape (e.g., a quadrilateral shape such as a square, rectangle, parallelogram, rhombus, etc.) and a fifth area that may be substantially identical to the first shape and the first area of the first cell 1011 and/or the third shape and the third area of the third cell 1013. In this way, the valve prosthesis 10 can be positioned such that the fifth cell 1088 may be aligned with one of the first ostia 810 or the second ostia 812 (e.g., possible ostia position indicated with dashed lines within the fifth cell 1088). For example, in aspects, the fifth cell 1088 can be aligned with one of the ostia 810, 812 such that a radial axis can intersect the longitudinal axis LA and pass through the fifth cell 1088 and the ostia 810, 812 aligned with the fifth cell 1088. As such, an unimpeded path can extend from the ostia and through the fifth cell 1088 along the radial axis. The unimpeded path comprises, for example, a cross-sectional shape and size that matches a cross-sectional shape and size of the ostia 810, 812 and the coronary arteries 809, 811, such that the unimpeded path does not intersect any of the struts 16, the commissures 901, 903, 905, or the prosthetic valve 20. In this way, the ostia 810, 812 are not blocked by the valve prosthesis 10 such that access to the ostia 810, 812 is provided to the coronary guide catheter 915 through the fifth cell 1088 along the unimpeded path.

In aspects, the prosthetic valve 20 can comprise a fifth edge segment 1092 and a sixth edge segment 1093 that are spaced apart and attached to the frame 15 at the first axial location 1017. By being attached to the frame 15, the edge segments 1092, 1093 can be attached to struts 16 of the frame 15, for example, by sutures or other mechanical fasteners. The fifth edge segment 1092 can be attached to, and extend toward, the second commissure 903, and the sixth edge segment 1093 can be attached to, and extend toward, the third commissure 905. The fifth cell 1088 can be positioned circumferentially between the fifth edge segment 1092 and the sixth edge segment 1093, with zero other edge segments between the fifth cell 1088 and the fifth edge segment 1092, and zero other edge segments between the fifth cell 1088 and the sixth edge segment 1093. In aspects, a fifth distance 1094 can circumferentially separate the fifth cell 1088 from the fifth edge segment 1092 at the first axial location 1017, and a sixth distance 1095 can circumferentially separate the fifth cell 1088 from the sixth edge segment 1093 at the first axial location 1017. In this way, the fifth cell 1088 is circumferentially offset from, and not at a midpoint between, the fifth edge segment 1092 and the sixth edge segment 1093. Likewise, the fifth cell 1088 is circumferentially offset from, and not at a midpoint between, the second commissure 903 and the third commissure 905. In aspects, the fifth edge segment 1092 can be circumferentially misaligned with, and offset from, the struts 16 at the first axial location 1017. For example, the fifth edge segment 1092 can be positioned between the closest struts to the fifth edge segment 1092 at the first axial location 1017. Likewise, the sixth edge segment 1093 can be circumferentially misaligned with, and offset from, the struts 16 at the first axial location 1017. For example, the sixth edge segment 1093 can be positioned between the closest struts to the sixth edge segment 1093 at the first axial location 1017.

Due to the attachment and position of the edge segments 1051, 1053, 1081, 1082, 1092, 1093 to the frame 15, the area that extends circumferentially between the commissures 901, 903, 905 at the first axial location 1017 may be non-symmetric. For example, at the first axial location 1017, a first region 1026 is defined between the first edge segment 1051 and the second edge segment 1053, a second region 1027 is defined between the third edge segment 1081 and the fourth edge segment 1082, and a third region 1028 is defined between the fifth edge segment 1092 and the sixth edge segment 1093. By being non-symmetric, the structure and shape of each of the regions 1026, 1027, 1028, for example, the position of the edge segments 1051, 1053, 1081, 1082, 1092, 1093 relative to the struts 16, is different. For example, the first region 1026 is bounded between the first edge segment 1051 (e.g., which is axially aligned with the first strut 1063) and the second edge segment 1053 (e.g., which bisects the struts 1071, 1073 and may be equidistant to the struts 1071, 1073). The second region 1027 is bounded between the third edge segment 1081 (e.g., which is not axially aligned with the closest struts, but, rather, is between and at differing distances from the closest struts) and the fourth edge segment 1082 (e.g., which is axially aligned with the strut 1085). In this way, the first region 1026 is different than, and non-symmetric to, the second region 1027, due to the location of the edge segments relative to the struts.

In addition, the position of the first cell 1011 relative to the edge segments 1051, 1053 differs from the position of the second cell 1023 relative to the edge segments 1081, 1082, for example, with the first distance 1057 different than the third distance 1083 and the fourth distance 1084, and the second distance 1059 different than the third distance 1083 and the fourth distance 1084. The third region 1028 is bounded between the fifth edge segment 1092 (e.g., which is not axially aligned with the closest struts, but, rather, is between and at differing distances from the closest struts) and the sixth edge segment 1093 (e.g., which is not axially aligned with the closest struts, but, rather, is between and at differing distances from the closest struts). In this way, the third region 1028 is different than, and non-symmetric to, the first region 1026 and the second region 1027, due to the location of the edge segments relative to the struts. Further, the position of the fifth cell 1088 relative to the edge segments 1092, 1093 differs from the position of the first cell 1011 relative to the edge segments 1051, 1053 and the second cell 1023 relative to the edge segments 1081, 1085. For example, the fifth distance 1094 and the sixth distance 1095 may each be different than each of the distances 1057, 1059 of the first cell 1011 and each of the distances 1083, 1084 of the second cell 1023.

In addition, or in the alternative, another way that the regions 1026, 1027, 1028 may be non-symmetric may be due to the circumferential length of each of the regions 1026, 1027, 1028. For example, a first circumferential length of the first region 1026 (e.g., distance between the edge segments 1051, 1053 along the first axial location 1017) may differ from one or both of a second circumferential length of the second region 1027 (e.g., distance between the edge segments 1081, 1082 along the first axial location 1017) or a third circumferential length of the third region 1028 (e.g., distance between the edge segments 1092, 1093 along the first axial location 1017). The circumferential lengths can be adjusted, for example, by providing additional cells between commissures 901, 903, 905 and/or by adjusting the attachment locations of the commissures 901, 903, 905 to the frame 15. As illustrated in FIG. 10, the first commissure 901 may lie within a cell (e.g., bounded by struts 16) while the second commissure 903 and the third commissure 905 may be attached to, and lie over, a junction or intersection of a plurality of struts. In aspects, a first cell distance 1097 can comprise a circumferential distance between the first cell 1011 and the second cell 1023, a second cell distance 1098 can comprise a circumferential distance between the second cell 1023 and the fifth cell 1088, and a third cell distance 1099 can comprise a circumferential distance between the first cell 1011 and the fifth cell 1088. In aspects, the cell distances 1097, 1098, 1099 may be different. For example, the first cell distance 1097 may be different than the second cell distance 1098 and the third cell distance 1099. The second cell distance 1098 may be different than the third cell distance 1099.

By providing the valve prosthesis 10 with the non-symmetric regions 1026, 1027, 1028, the valve prosthesis 10 can accommodate several different positions of, and circumferential separating distances between, the ostia 810, 812. For example, methods of rotationally aligning the transcatheter heart valve prosthesis 10 within a native heart valve (e.g., the treatment site 701) can initially comprise determining a position of the first ostia 810 and the second ostia 812 within the native heart valve. The position of the first ostia 810 and the second ostia 812 can comprise, for example, a circumferential separating distance 831 (e.g., illustrated in FIG. 8) between the first ostia 810 and the second ostia 812, an axial offset along the longitudinal axis LA (e.g., illustrated in FIG. 7) between the first coronary artery 809 and the second coronary artery 811, the location of the first ostia 810 and the second ostia 812 relative to other native structures within the treatment site 701, etc. In aspects, determining the position can occur prior to starting the procedure, for example, by an imaging modality such as a computed tomography (“CT”) scan. In this way, a physician can obtain information related to the positions of the ostia 810, 812 from the CT scan prior to determining a rotational orientation of the heart valve prosthesis 10 at the treatment site 701.

After determining the position of the first ostia 810 and the second ostia 812, methods can comprise selecting the transcatheter heart valve prosthesis 10 based on one or more of the first cell distance 1097 circumferentially separating the first cell 1011 from the second cell 1023, the second cell distance 1098 circumferentially separating the second cell 103 from the fifth cell 1088, or the third cell distance 1099 circumferentially separating the first cell 1011 from the fifth cell 1088. For example, the selection of a transcatheter heart valve prosthesis is based on a comparison between the determined positions of the first ostia 810 and the second ostia 812, and the dimensions of the valve prosthesis 10 to ensure that the valve prosthesis 10 can be positioned at the treatment site 701 without blocking the first ostia 810 or the second ostia 812. For example, the physician may also factor in some or all of the other distances 1035, 1037, 1043, 1057, 1059, 1083, 1084, 1094, 1095, 1097, 1098, 1099 described herein. In aspects, the valve prosthesis 10 can be positioned in three possible rotational orientations to align the commissures 901, 903, 905 with the native commissures 815, 817, 819 (e.g., illustrated in FIG. 8). For example, one possible rotational orientation comprises the first commissure 901, the second commissure 903, and the third commissure 905 being rotationally aligned (e.g., axially aligned with, overlapping, etc.) with the first native commissure 815, the second native commissure 817, and the third native commissure 819, respectively. A second possible rotational orientation comprises the first commissure 901, the second commissure 903, and the third commissure 905 rotationally aligned (e.g., axially aligned with, overlapping, etc.) with the second native commissure 817, the third native commissure 819, and the first native commissure 815, respectively. A third possible rotational orientation comprises the first commissure 901, the second commissure 903, and the third commissure 905 rotationally aligned (e.g., axially aligned with, overlapping, etc.) with the third native commissure 819, the first native commissure 815, and the second native commissure 817, respectively.

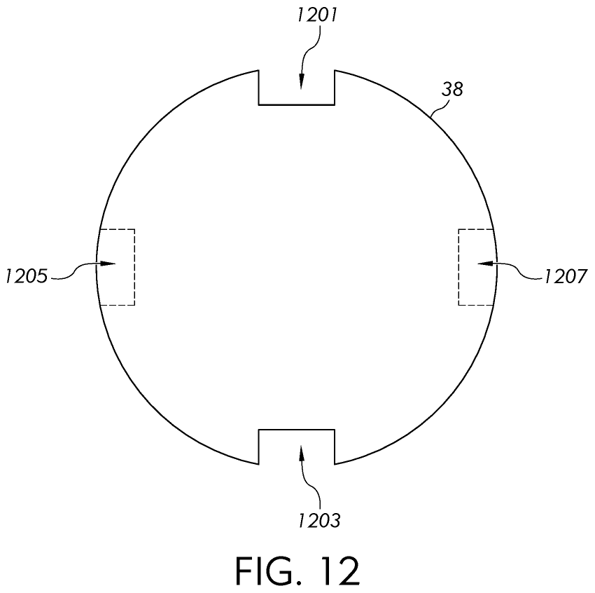

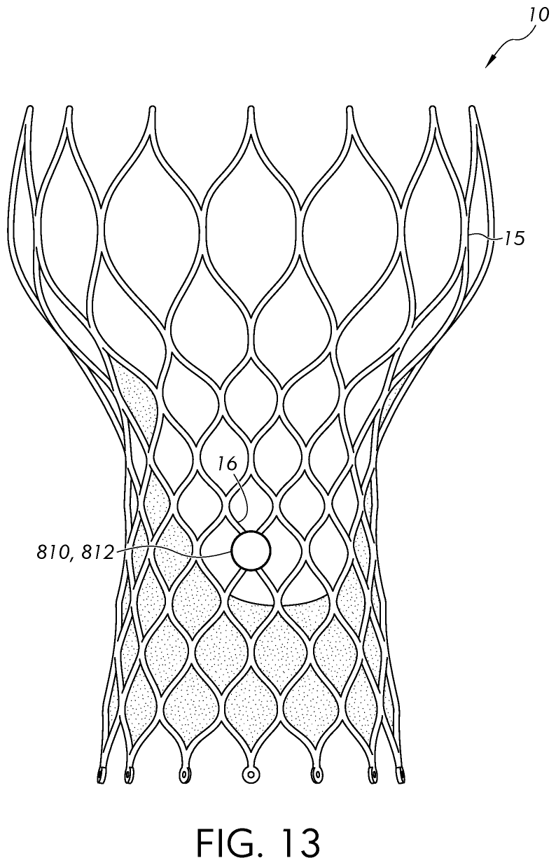

The physician can evaluate each of the three possible rotational orientations to determine whether each of the ostia 810, 812 can be aligned with one of the cells 1011, 1023, 1088 such that an unimpeded path may extend through the cells 1011, 1023, 1088 and to the ostia 810, 812. For example, the physician can first evaluate the first rotational orientation to determine if each of the ostia 810, 812 can be aligned with one of the cells 1011, 1023, 1088 such that an unimpeded path may be provided. If an unimpeded path is possible, then the physician may select the first rotational orientation of the valve prosthesis 10 and load the valve prosthesis 10 onto the delivery assembly 30 in a rotational orientation that allows the valve prosthesis 10 to be delivered to the treatment site 701 at the first rotational orientation. Conversely, if the physician determines that the first rotational orientation of the valve prosthesis 10 will not provide an unimpeded path to both the ostia 810, 812, then the physician can next evaluate the second rotational orientation in a similar manner. If an unimpeded path is possible in the second rotational orientation, then the physician may select the second rotational orientation of the valve prosthesis 10 and load the valve prosthesis 10 onto the delivery assembly 30 in a rotational orientation that allows the valve prosthesis 10 to be delivered to the treatment site 701 at the second rotational orientation. Conversely, if the physician determines that the second rotational orientation of the valve prosthesis 10 will not provide an unimpeded path to both the ostia 810, 812, then the physician can next evaluate the third rotational orientation in a similar manner. If an unimpeded path is possible in the third rotational orientation, then the physician may select the third rotational orientation of the valve prosthesis 10 and load the valve prosthesis 10 onto the delivery assembly 30 in a rotational orientation that allows the valve prosthesis 10 to be delivered to the treatment site 701 at the third rotational orientation. In this way, selecting the transcatheter heart valve prosthesis 10 can comprise not only selecting a valve that is suitable for the treatment site 701, but also selecting the best rotational orientation of the heart valve prosthesis 10 to provide the unimpeded paths to the ostia 810, 812. Accordingly, methods can comprise loading the transcatheter heart valve prosthesis 10 in a rotational orientation onto the delivery assembly 30, for example, the spindle 38 illustrated in FIG. 12, with the rotational orientation based on the position of the first ostia 810 and the second ostia 812.

In aspects, following the determination of the position of the ostia 810, 812 and selecting the heart valve prosthesis 10, methods can comprise percutaneously delivering the transcatheter heart valve prosthesis 10 to the treatment site 701 such that the first cell 1011 defined by one or more struts 16 is aligned with the first ostia 810 and a first unimpeded path extends from the first ostia 810 and through the first cell 1011, and the second cell 1023 defined by one or more of the struts 16 is aligned with the second ostia 812 and a second unimpeded path extends from the second ostia 812 and through the second cell 1023. Alternatively, in a different rotational orientation based on the position of the ostia 810, 812, the first cell 1011 can be aligned with the second ostia 812 and the fifth cell 1088 can be aligned with the first ostia 810. Further, in yet another alternative based on the position of the ostia 810, 812, the fifth cell 1088 can be aligned with the second ostia 812 and the second cell 1023 can be aligned with the first ostia 810. In aspects, percutaneously delivering the transcatheter heart valve prosthesis 10 to the treatment site 701 can comprise positioning one of the ostia 810, 812 circumferentially between two commissures 901, 903, 905 and circumferentially offset (e.g., closer to one commissure than another) relative to the two commissures 901, 903, 905. Alternatively, and based on the geometry, shape, and size of the heart valve prosthesis 10, percutaneously delivering the transcatheter heart valve prosthesis 10 to the treatment site 701 can comprise positioning one of the ostia 810, 812 circumferentially between two commissures 901, 903, 905 and circumferentially centered (e.g., equidistant to the two adjacent commissures) relative to the two commissures 901, 903, 905. In aspects, and to provide further unimpeded paths to the ostia 810, 812 through cells, the heart valve prosthesis 10 is not limited to being positioned such that the ostia 810, 812 are aligned with two of the first cell 1011, the second cell 1023, or the fifth cell 1088. Rather, in aspects, and, for example, when one ostia is axially offset from the other ostia, cells other than the first cell 1011, the second cell 1023, or the fifth cell 1088 can be aligned with one or both of the ostia 810, 812 to provide an unimpeded path to the ostia 810, 812. In aspects, following delivery of the heart valve prosthesis 10 to the treatment site 701, the heart valve prosthesis 10 can be rotated to a limited degree, for example, +/−5 degrees, to further assist in properly positioning the heart valve prosthesis 10.

FIG. 11 illustrates another embodiment of the heart valve prosthesis 10, wherein, in contrast to the cells of FIG. 10 in which substantially all of the cells comprise substantially the same shape and size, some cells may be larger than other cells. For example, one or more enlarged cells (e.g., access cells) may be provided which are of a sufficient size to be easily crossed with the coronary guide catheter 915 into either the first coronary artery 809 or the second coronary artery 811 once the heart valve prosthesis 10 has been deployed in situ. The heart valve prosthesis 10 can comprise a first cell 1101 positioned at substantially the same location as the first cell 1011 of FIG. 10, a second cell 1103 positioned at substantially the same location as the second cell 1023 of FIG. 10, and a fifth cell 1105 positioned at substantially the same location as the fifth cell 1088 of FIG. 10. In aspects, the first area of the first cell 1101 is different than the third area of the third cell 1013. For example, the first area can be at least double the third area or at least triple the third area. In aspects, the second area of the second cell 1103 is different than the fourth area of the fourth cell 1025. For example, the second area can be at least double the fourth area or at least triple the fourth area. In aspects, the first area may be different than the fourth area, for example, by being larger than the fourth area. In aspects, the fifth cell 1105 can comprise a fifth size and a fifth area that substantially matches the first cell 1101 and the second cell. In this way, due to the first cell 1101, the second cell 1103, and the fifth cell 1105 being larger than the surrounding cells, the first cell 1101, the second cell 1103, and the fifth cell 1105 can more easily be aligned with the ostia 810, 812 to provide the unimpeded paths.

FIG. 12 illustrates an end view of the spindle 38 of the delivery assembly 30 that can be attached to the heart valve prosthesis 10. In aspects, the spindle 38 can comprise one or more pockets that can receive the first paddle 1077 and the second paddle 1079. For example, the spindle 38 can comprise a first pocket 1201 and a second pocket 1203. The first pocket 1201 and the second pocket 1203 may be positioned substantially 180 degrees apart to match the distance (e.g., about 180 degrees) separating the first paddle 1077 from the second paddle 1079. The first pocket 1201 and the second pocket 1203 can be sized to receive the first paddle 1077 and the second paddle 1079. When the spindle 38 comprises two pockets 1201, 1203, there are two possible rotational orientations for the heart valve prosthesis 10: a first orientation in which the first paddle 1077 is received in the first pocket 1201 and the second paddle 1079 is received in the second pocket 1203, and a second orientation in which the first paddle 1077 is received in the second pocket 1203 and the second paddle 1079 is received in the first pocket 1201. However, in aspects, the spindle 38 may comprise additional pockets, for example, a third pocket 1205 and a fourth pocket 1207, to provide additional rotational orientations of the heart valve prosthesis 10. The third pocket 1205 and the fourth pocket 1207 are illustrated with dashed lines in FIG. 12 to show the variability in the total number of pockets in the spindle 38. The third pocket 1205 and the fourth pocket 1207 may be spaced about 90 degrees from the first pocket 1201 and the second pocket 1203, such that each of the pockets 1201, 1203, 1205, 1207 is spaced about 90 degrees from neighboring pockets. In this way, the spindle 38 can provide more than two rotational orientations for the heart valve prosthesis 10. In aspects, to provide additional rotational options, the spindle 38 may comprise any number of (e.g., two or more) pockets and/or the heart valve prosthesis 10 can comprise any number of (e.g., two or more) paddles, such that additional paddles could be provided. Two paddles and three pockets would result in three possible loading orientations. Two paddles and five pockets would result in five possible loading orientations.

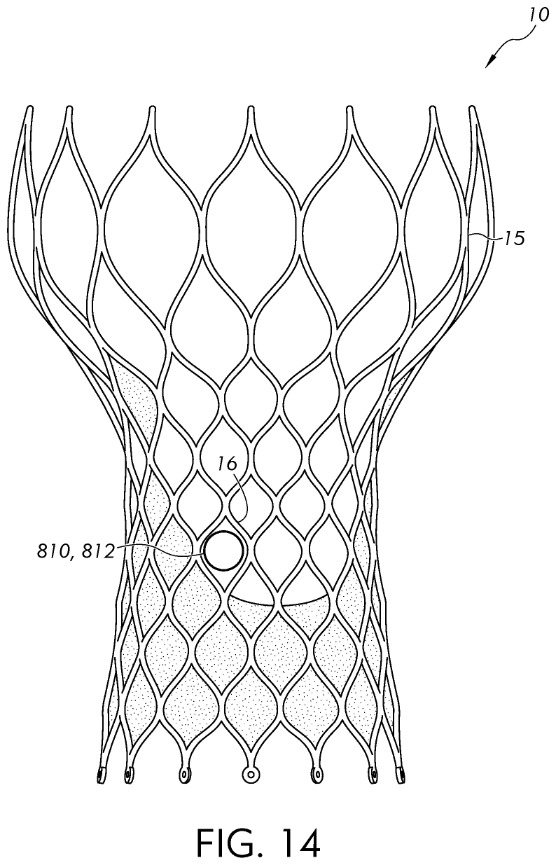

FIG. 13 illustrates a possible location of one of the ostia 810, 812 in which the ostia 810, 812 is obstructed by the struts 16. For example, as described herein, multiple rotational orientations may be provided in an effort to align the cells with the ostia 810, 812. However, in aspects, situations may arise in which the positions of the ostia 810, 812 are incompatible with the location of the cells, such that in any rotational orientation of the heart valve prosthesis 10, one or more struts 16 may obstruct one or both of the ostia 810, 812. To address this, selecting the transcatheter heart valve prosthesis 10 can further comprise altering the frame 15 of the transcatheter heart valve prosthesis 10 to adjust one or more of the first separating distance 1035, the second separating distance 1037, or the third separating distance 1043. For example, as illustrated in FIG. 14, altering the frame 15 can comprise altering a cell size of one or more of the first cell 1011, 1101, the second cell 1023, 1103, or the fifth cell 1088, 1105 to match an ostia size of one or more of the first ostia 810 or the second ostia 812. In this way, the shape of the cell that is aligned with the ostia can be altered and may be different than a shape of the surrounding cells. To further accommodate the positions of the ostia 810, 812, methods can comprise altering a position of the prosthetic valve 20 attached to the frame 15, for example, by shifting the position of the prosthetic valve 20 laterally or axially to allow for the ostia 810, 812 to be aligned with cells. In this way, a cell may be altered, for example, by manipulating or changing a position of the struts 16 that border the cell.

The heart valve prosthesis 10 disclosed herein can provide several benefits related to implantation of the heart valve prosthesis 10. For example, the heart valve prosthesis 10 comprises non-symmetric regions between commissures to accommodate for varying positions of the ostia. For example, the non-symmetric regions can accommodate at least three scenarios: mid-located ostia (e.g., at a midpoint between commissures), left-aligned ostia (e.g., in which the ostia is closer to one neighboring commissure than another neighboring commissure), or a right-aligned ostia (e.g., in which the ostia is closer to one neighboring commissure than another neighboring commissure). Further, the heart valve prosthesis 10 can be loaded onto a spindle in several different rotational loading orientations. In this way, a physician can determine the optimal deployed orientation of the heart valve prosthesis 10 to align with the native commissures and the ostia. Further, the physician can load the heart valve prosthesis 10 onto the spindle in a rotational orientation that will substantially match and allow the heart valve prosthesis 10 to be deployed in the desired deployed orientation at the treatment site. By initially loading the heart valve prosthesis 10 in the proper rotational orientation, manipulation and/or rotation of the heart valve prosthesis 10 after deployment of the heart valve prosthesis 10 can be avoided. Thus, the delivery and deployment of the heart valve prosthesis 10 can be simplified while also avoiding the obstruction of the ostia. To provide further variability, the heart valve prosthesis 10 can comprise non-constant shaped/sized cells, for example, access cells with a larger cross-sectional size to facilitate alignment with the ostia. Further, the cells can be varied in shape to accommodate the position of the ostia.

It should be understood that while various aspects have been described in detail relative to certain illustrative and specific examples thereof, the present disclosure should not be considered limited to such, as numerous modifications and combinations of the disclosed features are possible without departing from the scope of the following claims.

What is claimed is:

1. A transcatheter heart valve prosthesis comprising:
an annular frame extending along a longitudinal axis between an inflow end of the transcatheter heart valve prosthesis and an outflow end of the transcatheter heart valve prosthesis, the annular frame comprising a plurality of struts and configured to be adjustable between a radially-collapsed position and a radially-expanded position, the plurality of struts defining:
a first plurality of cells arranged along a first cell axis that is substantially parallel to the longitudinal axis, the first plurality of cells comprising a first cell at a first axial location, the first cell configured to be aligned with a first ostia such that a first radial axis intersects the longitudinal axis and passes through the first cell and the first ostia; and
a second plurality of cells arranged along a second cell axis that is substantially parallel to the longitudinal axis, the second plurality of cells spaced circumferentially apart from the first plurality of cells, the second plurality of cells comprising a second cell at the first axial location, the second cell configured to be aligned with a second ostia such that a second radial axis intersects the longitudinal axis and passes through the second cell and the second ostia; and
a prosthetic valve attached to the frame and comprising a plurality of leaflets, the prosthetic valve comprising a commissure where a first pair of leaflets of the plurality of leaflets are attached, the commissure attached to the frame and lying within a third cell axis that is substantially parallel to the longitudinal axis, the third cell axis positioned circumferentially between the first cell axis and the second cell axis such that a first separating distance circumferentially separates the first cell axis from the third cell axis and a second separating distance circumferentially separates the second cell axis from the third cell axis, the first separating distance different than the second separating distance.

2. The transcatheter heart valve prosthesis of claim 1, the prosthetic valve further comprising a second commissure where a second pair of leaflets of the plurality of leaflets are attached, the second commissure attached to the frame and lying within a fourth cell axis that is substantially parallel to the longitudinal axis, the first cell axis positioned circumferentially between the third cell axis and the fourth cell axis.

3. The transcatheter heart valve prosthesis of claim 2, wherein a third separating distance circumferentially separates the first cell axis from the fourth cell axis, the first separating distance different than the third separating distance.

4. The transcatheter heart valve prosthesis of claim 3, wherein the second separating distance is different than the third separating distance.

5. The transcatheter heart valve prosthesis of claim 4, further comprising a first paddle attached to the outflow end of the transcatheter heart valve prosthesis, the first paddle axially aligned with the commissure such that the first paddle lies within the third cell axis.

6. The transcatheter heart valve prosthesis of claim 5, further comprising a second paddle attached to the outflow end of the transcatheter heart valve prosthesis and spaced circumferentially apart from the first paddle, the second paddle axially misaligned from the second commissure.

7. A transcatheter heart valve prosthesis comprising:
an annular frame extending along a longitudinal axis between an inflow end of the transcatheter heart valve prosthesis and an outflow end of the transcatheter heart valve prosthesis, the annular frame comprising a plurality of struts and configured to be adjustable between a radially-collapsed position and a radially-expanded position, the plurality of struts defining:
a first plurality of cells arranged along a first cell axis that is substantially parallel to the longitudinal axis, the first plurality of cells comprising a first cell at a first axial location and a third cell at a second axial location spaced apart from the first axial location, the first cell comprising a first shape and a first area, and the third cell comprising a third shape and a third area, wherein one or more of the first shape is different than the third shape or the first area is different than the third area, the first cell configured to be aligned with a first ostia such that a first radial axis intersects the longitudinal axis and passes through the first cell and the first ostia; and
a second plurality of cells arranged along a second cell axis that is substantially parallel to the longitudinal axis, the second plurality of cells spaced circumferentially apart from the first plurality of cells, the second plurality of cells comprising a second cell at the first axial location and a fourth cell at the second axial location, the second cell comprising a second shape and a second area, and the fourth cell comprising a fourth shape and a fourth area, wherein one or more of the second shape is different than the fourth shape or the second area is different than the fourth area; and
a prosthetic valve attached to the frame and comprising one or more leaflets, the prosthetic valve comprising a first edge segment and a second edge segment spaced apart and attached to the frame at the first axial location, the first cell positioned circumferentially between the first edge segment and the second edge segment such that a first distance circumferentially separates the first cell from the first edge segment at the first axial location and a second distance circumferentially separates the first cell from the second edge segment at the first axial location, the first distance different than the second distance.

8. The transcatheter heart valve prosthesis of claim 7, wherein the second cell is configured to be aligned with a second ostia such that a second radial axis intersects the longitudinal axis and passes through the second cell and the second ostia.

9. The transcatheter heart valve prosthesis of claim 8, wherein the first shape is substantially identical to the second shape and the first area is substantially identical to the second area.

10. The transcatheter heart valve prosthesis of claim 9, wherein one or more of the first shape is different than the fourth shape or the first area is different than the fourth area.

11. The transcatheter heart valve prosthesis of claim 7, wherein the first edge segment is circumferentially aligned with a first strut of the plurality of struts at the first axial location, the first strut lying between two cells at the first axial location.

12. The transcatheter heart valve prosthesis of claim 11, wherein the second edge segment is circumferentially misaligned with and offset from the plurality of struts at the first axial location.

13. A method of rotationally aligning a transcatheter heart valve prosthesis within a native heart valve, the method comprising:
determining a position of a first ostia and a second ostia within the native heart valve;
loading the transcatheter heart valve prosthesis in a first rotational orientation onto a delivery assembly, the first rotational orientation based on the position of the first ostia and the second ostia;
percutaneously delivering the transcatheter heart valve prosthesis to the native heart valve such that a first cell defined by one or more struts of the transcatheter heart valve prosthesis is aligned with the first ostia and a first unimpeded path extends from the first ostia and through the first cell, and a second cell defined by one or more of the struts is aligned with the second ostia and a second unimpeded path extends from the second ostia and through the second cell.

14. The method of claim 13, wherein after determining the position and before loading the transcatheter heart valve prosthesis, further comprising selecting the transcatheter heart valve prosthesis based on one or more of:
a first cell distance circumferentially separating the first cell from the second cell;
a second cell distance circumferentially separating the second cell from a third cell; or
a third cell distance circumferentially separating the first cell from the third cell.

15. The method of claim 14, wherein selecting the transcatheter heart valve prosthesis further comprises altering a frame of the transcatheter heart valve prosthesis to adjust one or more of the first separating distance, the second separating distance, or the third separating distance.

16. The method of claim 14, wherein selecting the transcatheter heart valve prosthesis further comprises altering a position of a prosthetic valve attached to the frame.

17. The method of claim 14, wherein selecting the transcatheter heart valve prosthesis further comprises altering a cell size of one or more of the first cell, the second cell, or the third cell to match an ostia size of one or more of the first ostia or the second ostia.

18. The method of claim 13, wherein percutaneously delivering the transcatheter heart valve prosthesis comprises positioning the first ostia circumferentially between two commissures and circumferentially centered relative to the two commissures.

19. The method of claim 18, wherein percutaneously delivering the transcatheter heart valve prosthesis comprises positioning the second ostia circumferentially between two commissures and circumferentially offset relative to the two commissures.

20. The method of claim 13, wherein the first unimpeded path and the second unimpeded path do not intersect any of the one or more struts.
